(19) United States
(12) Patent Application Publication
(10) Pub. No.: US 2015/0147412 A1
(43) Pub. Date: May 28, 2015

(54) Sulfuric Acid Burn Treatment Composition

(71) Applicant: Mark Greene, Perrysburg, OH (US)

(72) Inventor: Mark Greene, Perrysburg, OH (US)

(21) Appl. No.: 14/547,906

(22) Filed: Nov. 19, 2014

Related U.S. Application Data
(60) Provisional application No. 61/907,657, filed on Nov. 22, 2013.

(51) Int. Cl. A61K 36/9066; A61K 31/7004; A61K 31/047; A61K 33/40; A61K 31/05; A61K 31/085; A61K 36/15; A61K 33/08; A61K 36/886

(52) U.S. Cl. CPC A61K 36/9066; A61K 33/08; A61K 31/7004; A61K 31/047; A61K 36/886; A61K 31/05; A61K 31/085; A61K 36/15; A61K 33/40
USPC 424/693; 424/744

(57) ABSTRACT

Disclosed is a composition for treating sulfuric acid burn, wherein the composition may be used as a topical medication. The composition generally includes isopropyl alcohol or L-valine solution; at least one active therapeutic substance and/or a disinfectant for treating the burn; calcium hydroxide for neutralizing the acid; a humectant and/or a clinging agent for preventing the acid from spreading; an expectorant for signaling the body to clear any irritations caused by the acid; an acid indicator for visually signaling presence of the acid; and fumed silica as a thickening agent for slowing the reaction time of the acid. In a preferred embodiment, and without limitation, the therapeutic substances may include eugenol and thymol, and the disinfectants may include turmeric powder and hydrogen peroxide. Additionally, glycerin and sugar are used as clinging agents to reduce the chemical reaction between the sulfuric acid and surrounding tissue or materials exposed thereto.

### CROSS REFERENCE TO RELATED APPLICATION

This application claims the benefit of U.S. Provisional Application No. 61/907,657 filed on Nov. 22, 2013. The above identified patent application is herein incorporated by reference in its entirety to provide continuity of disclosure.

### BACKGROUND OF THE INVENTION

1. Field of the Invention

The present invention relates to a composition for treating chemical burns. More specifically, the present invention pertains to an improved composition for treating burns from sulfuric acid, wherein the composition comprises calcium hydroxide as the active ingredient for neutralizing sulfuric acid. The present composition may be in a liquid form or a semi-liquid form, depending upon embodiment, and is suitable for use as a topical medication.

Sulfuric acid is a highly corrosive, strong mineral acid. Sulfuric acid has various applications and can be used as a domestic acidic drain cleaner, an electrolyte in lead-acid batteries, and as a cleaning agent. It is also a central substance in the chemical industry. Principal uses include mineral processing, fertilizer manufacturing, oil refining, wastewater processing, and chemical synthesis.

Sulfuric acid at a high concentration can cause very serious damage upon contact with skin or eyes, as it not only causes chemical burns via hydrolysis, but also secondary thermal burns via dehydration. Upon contact with eyes, it burns the cornea and can lead to permanent blindness. Accordingly, safety precautions should be strictly observed when handling sulfuric acid. Still, many individuals may be exposed to uncontained sulfuric acid through spills and other accidents.

Sulfuric acid burns, like most chemical burns, are generally treated first by rinsing or flushing the chemical off of a person's body with a large amount of cool water. It is important to treat the burn correctly to avoid further complications. Additionally, sulfuric acid can be flushed with a mild, soapy solution if the burns are not severe. Sulfuric acid feels hot when water is added to the acid, but it is better to flush the area and remove the acid from the skin.

The present invention provides a composition for treating sulfuric acid burns. The composition of the present invention is adapted to neutralize the sulfuric acid so as to alleviate the burn or other types of damage caused thereby. In one embodiment, the composition of the present invention is adapted to treat external burns to the body. A preferred embodiment of the present composition comprises isopropyl alcohol, calcium hydroxide or calcium carbonate, sugar, turmeric powder, glycerin, oil of turpentine, fumed silica, and hydrogen peroxide. In another embodiment, L-valine solution may be used in lieu of isopropyl alcohol. If L-valine solution is used, one or more therapeutic substances such as eugenol and thymol, as well as a soothing agent such as aloe vera gel may be added. The hydroxide base from calcium hydroxide can react with the hydrogen ions in the sulfuric acid to form water, thereby neutralizing the acid.

Other embodiments of the present invention may further include baking soda for treating damage to the ears, and tea tree oil for treating damage to the eyes. In extreme circumstances where sulfuric acid is inhaled or swallowed, one embodiment of the present invention may be administered by mouth or nose to treat burns to the nose, esophagus, and stomach. Such embodiments may comprise L-valine solution, calcium carbonate, powdered sugar, turmeric powder, aloe vera gel, glycerin, eugenol, and thymol.

2. Description of the Prior Art

Calcium hydroxide and calcium carbonate have been disclosed in the prior art as widely used active ingredients for compositions used to neutralize an acid, to treat liquid waste or sewage, and to treat potable water. None of the prior art references, however, disclose a composition for treating sulfuric acid burn, wherein the composition comprises sugar, oil of turpentine, hydrogen peroxide, and turmeric powder. Moreover, none of the prior art references disclose a composition for treating sulfuric acid burn to the eyes, ears, esophagus, and stomach. The foregoing is a list of prior art compositions deemed most relevant to the present disclosure, which are herein described for the purposes of highlighting and differentiating the unique aspects of the present invention, and further highlighting the drawbacks existing in the prior art.

Specifically, U.S. Pat. No. 5,326,549 to Miyata discloses bimetal hydroxide for neutralizing or inactivating acids or halogens in plastics and rubbers. The bimetal hydroxide has the formula: Caₓ₋₁M²⁺ₓ(OH)₂, wherein M²⁺ is at least one divalent metal ion selected from the group consisting of Mg²⁺, Mn²⁺, Fe²⁺, Co²⁺, Ni²⁺, Cu²⁺, Zn²⁺, and wherein x is defined by 0.005<x <0.4. The present invention, however, does not comprise a bimetal hydroxide. Rather, the present invention discloses a hydroxide or carbonate base that reacts with the hydrogen ions in the acid to form water, neutralizing sulfuric acid and thereby reducing burns and damages caused thereby.

U.S. Published patent application No. 2007/0082978 to Suzuki discloses a resin composition comprising calcium hydroxide. Calcium hydroxide has a high thermal stability and is used to stabilize synthetic resins. Suzuki, however, does not disclose a composition containing oil of turpentine, hydrogen peroxide, and turmeric powder. Additionally, Suzuki does not disclose similar therapeutic substance and/or a disinfectant for treating the burn caused from sulfuric acid.

U.S. Published patent application No. 2003/0121457 to Butters discloses a stabilized calcium hydroxide slurry containing calcium hydroxide, at least one acidic polymer or water-soluble salt, at least one co-additive selected from boric acid, and water soluble salts of boric acid. The present invention, however, does not include at least one co-additive selected from boric acid or water soluble salts of boric acid.

Finally, U.S. Published patent application No. 2013/0126116 to Solismaa discloses a method and a system for precipitation of calcium carbonate and a product comprising calcium carbonate. However, Solismaa does not disclose a method and a system for neutralizing sulfuric acid. Additionally, the present invention does not precipitate calcium carbonate. Rather, calcium carbonate is used to release water and carbon dioxide.

The prior art compositions differ from the present invention in that none of the prior art references disclose a composition containing isopropyl alcohol or L-valine solution, calcium hydroxide or calcium carbonate, sugar, glycerin, fumed silica, oil of turpentine, hydrogen peroxide, and turmeric powder. Additionally, the prior art compositions do not include disinfectant such as eugenol and thymol for alleviating burns caused by sulfuric acid. Inclusion of such therapeutic substances and/or disinfectants is advantageous in that these substances can inhibit growth of microorganisms that can cause infections to the burned skin.

It is therefore submitted that the present invention is substantially divergent in design elements from the prior art, and consequently it is clear that there is a need in the art for an improvement to compositions used to neutralize an acid. In this regard, the instant invention substantially fulfills these needs.

### SUMMARY OF THE INVENTION

In view of the foregoing disadvantages inherent in the known types of sulfuric acid burn treatment compositions now present in the prior art, the present invention provides a new and improved sulfuric acid burn treatment composition wherein the same can be utilized for neutralizing sulfuric acid and mitigating the damage caused thereby.

It is therefore an object of the invention to provide a new and improved sulfuric acid burn treatment composition that has all of the advantages of the prior art and none of the disadvantages.

Another object of the present invention is to provide a new and improved sulfuric acid burn treatment composition having a solvent, at least one active therapeutic substance and/or a disinfectant, a strong base, a humectant and/or a clinging agent, an expectorant, an acid indicator, and a thickening agent.

Yet another object of the present invention is to provide a new and improved sulfuric acid burn treatment composition comprising calcium hydroxide, powdered sugar, glycerin, oil of turpentine, hydrogen peroxide, turmeric powder, and isopropyl alcohol or L-valine in water, and/or fumed silica, depending upon embodiment, for external burns

Still yet another object of the present invention is to provide a new and improved sulfuric acid burn treatment composition comprising isopropyl alcohol, baking soda, turmeric powder, aloe vera gel, and glycerin for treating burns to the ears.

Still yet another object of the present invention is to provide a new and improved sulfuric acid burn treatment composition comprising a L-valine solution, calcium carbonate, turmeric powder, aloe vera gel, glycerin, tea tree oil, eugenol, and thymol for treating burns to the eyes.

Still yet another object of the present invention is to provide a new and improved sulfuric acid burn treatment comprising a L-valine solution, calcium carbonate, powdered sugar, turmeric powder, aloe vera gel, glycerin, eugenol, and thymol, for treating burns to the esophagus and stomach.

Still yet another object of the present invention is to provide a new and improved sulfuric acid burn treatment composition that is safe for use as a topical medication.

Still yet another object of the present invention is to provide a new and improved sulfuric acid burn treatment composition in a form of liquid or in a form of semi-liquid mixture.

Still yet another object of the present invention is to provide a new and improved sulfuric acid burn treatment composition wherein the device may be readily fabricated from materials that permit relative economy and are commensurate with durability.

Other objects, features, and advantages of the present invention will become apparent from the following detailed description taken in conjunction with the accompanying drawings.

### DETAILED DESCRIPTION OF THE INVENTION

For the purposes of presenting a brief and clear description of the present invention, the preferred embodiment will be discussed as used to neutralize sulfuric acid and mitigate the damage caused thereby. The present invention comprises a composition for treating burns caused by sulfuric acid. The present composition may be in the form of liquid or in the form of semi-liquid mixture, such as cream or ointment. The composition generally contains: a solvent; at least one active therapeutic substance and/or a disinfectant for treating the burn; a strong base for neutralizing the acid; a humectant and/or a clinging agent for preventing the acid from spreading; an expectorant for signaling the body to increase secretion to clear any irritations caused by the acid; an acid indicator for visually signaling presence of the acid; and a thickening agent for slowing the reaction time of the acid.

In one embodiment, the solvent comprises 70% isopropyl alcohol. Isopropyl alcohol is stable against strong acid such as sulfuric acid. Isopropyl alcohol is a preferred solvent for the present composition because isopropyl alcohol is widely used as a solvent and as an antibacterial cleaning liquid. Alternatively, it is contemplated that propylene glycol or L-valine solution may be used in lieu of isopropyl alcohol because propylene glycol, L-valine, and isopropyl alcohol share similar chemical properties.

L-valine solution is composed of L-valine diluted in distilled water. L-valine solution, however, does not comprise antibacterial properties. Thus, at least one active therapeutic substance such as eugenol and thymol may be added to provide antibacterial effects. While eugenol and/or thymol may primarily be added to compositions containing L-valine solution in lieu of isopropyl alcohol, these therapeutic substances may also be added to compositions containing isopropyl alcohol as shown in TABLE 1. Further, eugenol and/or thymol may be used in lieu of other therapeutic substances. It is contemplated that the following compositions may be utilized to treat external burns to the body.

Other therapeutic substances and/or disinfectants comprise oil of turpentine, turmeric powder, aloe vera, and hydrogen peroxide. Oil of turpentine yields terebene when mixed with sulfuric acid. Terebene is a known antiseptic, but it can also help break down clumps of sulfuric acid. In this way, terebene can be used as an expectorant to clear any irritations from the body that is caused by the acid. Turmeric powder also has antiseptic, as well as anti-inflammatory properties. Turmeric powder also changes color in the presence of acid. As such, turmeric powder can be used to visually detect the presence of sulfuric acid, and the acid burn can be treated until the acid is no longer present in the affected area.

The present invention further comprises calcium hydroxide, which is used as a strong base. In some embodiments, however, calcium hydroxide may be substituted for calcium carbonate. If calcium hydroxide is used, carbon dioxide can be added to form calcium carbonate and water. Calcium hydroxide can react with sulfuric acid to produce water, while neutralizing the sulfuric acid by raising the pH level. Though calcium hydroxide is not completely soluble in isopropyl alcohol, it is soluble in glycerin, which acts as a clinging agent as well as a humectant. Glycerin acts to retain moisture, and because sulfuric acid has a high affinity for water, sulfuric acid clings to glycerin. In this way, glycerin prevents the spread of the acid that would normally dilute and spread. Further, powdered sugar acts as a supplemental clinging agent when exposed to sulfuric acid, and can help slow the reaction time of sulfuric acid, thereby reducing the amount of damage caused by the acid when it is exposed to the body.

Optionally, a thickening agent can be added to thicken the composition so that the composition is in a form of a semi-liquid mixture such as cream or an ointment. In a preferred embodiment, fumed silica is used as a thickening agent. Fumed silica is highly stable and does not easily react with acid. Additionally, fumed silica has a high solubility and thickening efficiency. In this way, fumed silica is an ideal thickening agent for use with a strong acid such as sulfuric acid. As the amount of fumed silica added is increased, the viscosity of the composition increases, which prevents sulfuric acid from spreading quickly, and causes the reaction time of the sulfuric acid to decrease. It is contemplated that in a preferred embodiment, the ratio of fumed silica to the composition is approximately 2:1 by volume.

TABLE 1

| Ingredient | Example 1 | Example 2 | Example 3 |
|---|---|---|---|
| Solvent | 8 oz. of 70% | 8 oz. of 70% | 40 g L-valine |
| | isopropyl alcohol | isopropyl alcohol | diluted in 8 oz. |
| | and fumed | and fumed | of distilled water |
| | silica for | silica for | and fumed |
| | thickening | thickening | silica for |
| | | | thickening |
| Ca(OH)₂ | 1 Tbsp. | 2 Tbsp. | 2 Tbsp. |
| Aloe Vera Gel | — | ½ tsp. | ½ tsp. |
| Powdered | ⅛ tsp. | ⅛ tsp. | ⅛ tsp. |
| Sugar | | | |
| Turmeric | ⅛ tsp. | 1/16 tsp. | 1/16 tsp. |
| Powder | | | |
| Thymol | — | 0.0125 g | 0.0125 g |
| Eugenol | — | 0.0125 g | 0.0125 g |
| Glycerin | 0.0625 oz. | 0.0625 oz. | 0.0625 oz. |
| Oil of | 0.001 oz. | — | — |
| Turpentine | | | |
| Hydrogen | 0.001 oz. | — | — |
| Peroxide | | | |

Another embodiment of the present invention may be suitable for treating injuries to the ears. Such composition includes: 4 oz. of 70% isopropyl alcohol; 1/16 tsp. of baking soda; 1/16 tsp. of turmeric powder; ½ tsp. of aloe vera gel; and 1 v/v % of glycerin. In this embodiment, isopropyl alcohol helps to dry the ears, while the baking soda helps drive the composition and the sulfuric acid out of the ears.

Another embodiment may be suitable for treating injuries to the eyes. Such composition includes: 10 g of L-valine dissolved in 2 oz. of distilled water; 1/16 tsp. of calcium carbonate; 1/16 tsp. of turmeric powder; ½ tsp. of aloe vera gel; 1 v/v % of glycerin; 20 v/v % of tea tree oil; 0.025 g of eugenol; 0.0125 g of 0.051% thymol.

Yet another embodiment of the present composition may be ingested when an individual has swallowed or inhaled sulfuric acid, and the individual has burns in the nose, esophagus, and the stomach. Such composition comprises: 40 g of L-valine diluted in 8 oz. of distilled water; 3 Tbsp. of calcium carbonate; ⅛ tsp. of powdered sugar; 1/16 tsp. of turmeric powder; ½ tsp. of aloe vera gel; 0.0625 oz. of glycerin; 0.025 g of eugenol; 0.0125 g of thymol. The foregoing composition may be preferably be in a liquid form so that it can be sprayed into the nostril. However, fumed silica may be added to so that the composition can be thickened and used as an ointment.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A composition for treating sulfuric acid burns, comprising:
approximately 8 ounces of solvent;
approximately 1 tablespoon to 2 tablespoons of calcium hydroxide;
approximately ⅛ teaspoon of powdered sugar;
approximately 1/16 teaspoon to ⅛ teaspoon of turmeric powder, said turmeric powder adapted to change color in a presence of an acid;
approximately 0.0625 ounces of glycerin.

2. The composition for treating sulfuric acid burns of claim 1, wherein said solvent comprises 70% isopropyl alcohol.

3. The composition for treating sulfuric acid burns of claim 1, further comprising:
fumed silica, wherein a ratio of fumed silica to a mixture containing said solvent, said calcium carbonate, said powdered sugar, said turmeric powder, and said glycerin is approximately 2:1 by volume.

4. The composition for treating sulfuric acid burns of claim 3, further comprising:
approximately 0.001 ounces of oil of turpentine;
approximately 0.001 ounces of hydrogen peroxide.

5. The composition for treating sulfuric acid burns of claim 1, further comprising:
approximately ½ teaspoon of aloe vera gel;
approximately 0.0125 grams of thymol;
approximately 0.0125 grams of eugenol; and
wherein said solvent comprises 40 grams of L-valine.

6. A composition for treating sulfuric acid burns, comprising:
solvent;
1/16 teaspoon of turmeric powder, said turmeric powder changing colors in a presence of an acid;
½ teaspoon of aloe vera gel;
at least one clinging agent.

7. The composition for treating sulfuric acid burns of claim 6, further comprising 1/16 teaspoon of baking soda;
wherein said at least one clinging agent comprises 1 v/v % of glycerin; and
wherein said solvent comprises 4 ounces of 70% isopropyl alcohol.

8. The composition for treating sulfuric acid burns of claim 6, further comprising 0.0125 grams of thymol and 0.025 grams of eugenol.

9. The composition for treating sulfuric acid burns of claim 8, further comprising 1/16 teaspoon of calcium carbonate;
wherein said at least one clinging agent comprises 1 v/v % of glycerin;
20 v/v % of tea tree oil; and
wherein said solvent comprises 10 grams of L-valine dissolved in 2 ounces of distilled water.

10. The composition for treating sulfuric acid burns of claim 8, further comprising 3 tablespoons of calcium carbonate;
wherein said at least one clinging agent comprises 0.0625 ounces of glycerina and ⅛ teaspoon of powdered sugar; and
wherein said solvent comprises 40 grams of L-valine diluted in 8 ounces of distilled water.
